(19) United States
(12) Patent Application Publication
(10) Pub. No.: US 2025/0082202 A1
(43) Pub. Date: Mar. 13, 2025

(54) Endoscope Having a Dual Camera Module

(71) Applicant: Retivue, LLC, Charlottesville, VA (US)

(72) Inventors: Paul Yates, Charlottesville, VA (US); Viatcheslav Orlov, Charlottesville, VA (US); Nelson Planting, Charlottesville, VA (US)

(21) Appl. No.: 18/641,311

(22) Filed: Apr. 19, 2024

Related U.S. Application Data
(60) Provisional application No. 63/497,037, filed on Apr. 19, 2023.

(51) Int. Cl. A61B 3/14

(52) U.S. Cl. CPC A61B 3/145

(57) ABSTRACT

Dual camera module ophthalmic endoscope is disclosed that can fully recapitulate the traditional stereo view of said retina of said eye provided thru a surgical microscope when said vitreoretinal surgeon is viewing said retina using said surgical microscope, looking thru said cornea and said lens of said eye. Said dual camera module ophthalmic endoscope is comprised of: 1) an ultra-small camera sensor to directly image said retina of said eye providing higher resolution image from said camera modules than a commercial endoscope using fiber optic rather than direct ultra-small camera sensor to image said retina 2) an ultra-wide 150° field of view lens system paired with said camera sensor to provide full imaging of said retina to said ora serata, from a central, macula-directed positioning of said endoscope camera module in said eye 3) fiber optic illumination system to fully illuminate said 150° field of view imaged by said ultra-wide 150° field of view lens system, 4) software algorithm to integrate said dual images provided by said dual camera modules to provide said vitreoretinal surgeon a 3-D, stereoscopic heads-up, top-down view of the retina using said dual camera module ophthalmic endoscope.

### CLAIM OF PRIORITY

This application claims benefit of priority of U.S. Provisional Patent Application Number 63/497,037, Yates et al., titled “Endoscope Having a Dual Camera Module,” filed on Apr. 19, 2023, which is hereby incorporated by reference herein in its entirety.

### TECHNICAL FIELD

The present subject matter relates to an endoscope for imaging the retina of the eye.

### BACKGROUND

Vitreoretinal surgeons require an unobstructed view thru cornea (12) and lens (14) of the eye (107) to allow the visualization necessary for fine manipulation of retinal tissue. Unfortunately, many patients have pre-existing obscurations or develop clouding of the view intra-operatively (FIG. 2) that frequently prevents addressing complex retinal pathology such as proliferative diabetic retinopathy¹⁻⁴ . Ophthalmic endoscopes, inserted thru the sclera (17, 32) to bypass a cloudy cornea, have existed for 30 years^{5, 6} but been poorly adopted due to 1) a disorienting, non-top down, often rotated view⁵ (18) 2) a large learning curve to become facile with probe movement⁷ 3) inability to perform bi-manual surgery due to holding the endoscope for both viewing and illumination⁸ (FIG. 1) 4) dissociation of surgeon hand movement from image perception⁹ 5) lack of stereopsis for gauging depth¹⁰ with a flat non-stereo LCD display showing the retina (FIG. 3, 31) 6) high cost and 7) very low resolution (<17K pixels)¹⁰ limiting retinal detail.

### SUMMARY

The present invention discloses endoscopic visualization for use in ophthalmic applications to provides a hands-free (FIG. 13, 14), low cost, higher resolution (FIG. 19), standardized heads-up (16), correct perspective (FIG. 6), stereo view of the retina (FIG. 17). A preferred embodiment of this invention replicates a standard top-down¹¹ view (16) used in vitreoretinal surgery to enhance surgeon adoption of this endoscopic approach for surgical visualization to improve patient outcomes. An additional aspect of this preferred embodiment incorporates a low-cost, disposable endoscopic (FIG. 4, 43) approach that is able to fully recapitulate the traditional surgical microscope top-down view (FIG. 5) provided thru cornea and lens (12, 14). As disclosed in this specification a preferred embodiment of the disclosed invention may consist of at least one of the following capabilities:
1) a low cost, disposable, dual camera (FIG. 13, 14) module system (43, 104), anchored to sclera (33) thru 19-20 g sclerotomies.
2) said camera module (104) providing hands-free, stereo (FIG. 17) view of the retina for both macro <60° and widefield >60° field of view (FOV) (FIG. 5, 13, 14)
3) said camera module having variable depth of insertion to provide different field of view (FOV)
4) said camera module (104) having adjustable positioning within said eye to visualize the ora serrata and the macula of said retina of said eye when said camera module position is adjusted (FIG. 13, 14).
5) video processing (FIG. 12) of camera images provided by said camera module or modules to accomplish at least one of:
   5.a) enhance image resolution of said camera images to improve resolution of said retina (FIG. 7)
   5.b) correction in said camera images of warped side-view perspective (FIG. 6) provided by said camera module to enable a top down (16) perspective of said retina to simulate viewing top-down thru said cornea of said eye (FIG. 1, 16).
   5.c) combination of said dual camera images (FIG. 12) from said dual camera modules to provide stereopsis (FIG. 17) to said dual camera (FIG. 13, 14) images when viewing said dual camera images, allow for true depth feedback in a heads-up display for the surgeon.
6) synchronized repositioning of camera module tilt (FIG. 10, 11, 13, 14) and direction when rotating the eye, to properly reconnect hand movement to image perception, replicating the expected top-down view (FIG. 6) when the eye is rotated.

Over 300,000 retinal surgeries are performed in the United States each year¹². Successful outcomes for ophthalmic surgery have been predicated for the last 50 years on the surgeon having a clear, top-down (16), 3-D stereo view of the eye for surgical procedures, be it removal of cataracts from the anterior segment, or repair of retinal pathology in the posterior segment¹. The eye, at less than 25 mm diameter, 8 cm³ is a highly confined space, yet is routinely amenable to fine microsurgical procedures with 27 g (<0.5 mm diameter) instrumentation, manipulating micron thickness pathologic tissue to help restore vision. Training requires years of practice to gain the muscle memory and a microsurgical skill set to consistently achieve successful restoration of vision for patients with retinal disease¹³.

A frequent difficulty for the vitreoretinal surgeon is that a top-down (16) oriented view is not always available, limited from the start due to pre-existing corneal and lenticular opacifications (FIG. 2), or becoming progressively hazy during the surgical procedure itself (i.e. from hydration of the cornea, formation of corneal Descemet's folds, or fogging of the lens due to lens composition and irregular fluid-air interfaces)^{1, 5}. The vitreoretinal surgeon is often faced in these situations with the choice to postpone time-critical surgeries (i.e. ocular trauma, endophthalmitis, retinal detachment, acute glaucoma) due to this suboptimal viewing, hoping for a better view in days or weeks, performing an invasive and time-consuming penetrating keratoplasty with temporary keratoprosthesis to allow the retina to be seen¹, or limiting the extent of what can be surgically addressed due to an inability to visualize the retina with good detail. Retinal surgeons must frequently make this decision in the midst of the surgery itself. Up to 30% of vitreoretinal surgeries encounter viewing issues either pre- or intra-operatively. Unfortunately, the suboptimal options in these instances limit operative time, negatively impact surgical outcomes, and ultimately reduce recovery of vision for patients.

An endoscopic solution for retinal viewing was introduced by Uram (FIG. 1, FIG. 3) in the 1990s¹⁴. A handheld probe (17) is inserted thru the (33) pars plana, providing both fiber-optic illumination and simultaneous viewing of the retina (18), (FIG. 19). Despite routine availability of these devices, and even with successive iterations that have made it compatible with smaller 25 and 27 gauge surgical instrumentation, it is rarely employed to this day. The lack of adoption is multi-factorial, but chief among most surgeons concerns are non-intuitive use, and a highly disorienting view that these devices provide compared to traditional ophthalmic viewing systems⁵.

Additional challenges occur when using an ophthalmic endoscope: 1) routine bi-manual retinal surgery is eliminated as the endoscopic probe must be held away from the retina for viewing (FIG. 1, FIG. 3). 2) a variably magnified side (18), rather than top-down (16), view is provided, with the probe frequently moved closer to the retina (18) to better illuminate the area of interest, simultaneously collapsing the field of view⁹ 3) maintaining probe orientation and rotation becomes critical to know up from down 4) the view provided disconnects surgeon fine stereotactic instrument movements from the 2D image perception provided on-screen (FIG. 3, 31). 5) stereopsis is fully lost with a flat 2D image on the screen (31, 191, 192), rendering microsurgical tissue manipulation extremely difficult 6) resolution is poor, with as few as 6000 pixel images provided for 27 gauge surgeries (191, 192). ¹⁰ 7) focus is manual and depth limited 8) viewing is off to the side of the operating microscope on a low resolution CRT (FIG. 3). These obstacles can be overcome with substantial training, creating new muscle memory, but given how foreign this mode of operation is from everyday surgery, it remains infrequently practiced despite the benefit having such skills could provide.

Despite this aversion to ophthalmic endoscopes, vitreoretinal surgeons have readily adopted heads-up viewing¹¹ systems for vitreoretinal surgery. The ability to provide real-time, dynamic enhancement of the direct image available thru a surgical microscope has been shown to have good utility for completing procedures¹¹. Most importantly there is a substantially lower threshold to incorporation of this mode of remote viewing into standard vitreoretinal surgical routine, as the relative top-down view (16) is preserved, despite the provided image being camera sensor based, rather than direct viewing of the retina with the surgeons own eyes. Thus, a likely key challenge for wider adoption of remote ophthalmic endoscopic systems is to better enable a modality of viewing with greater similarity to standard operative procedures (FIG. 6).

While ophthalmic endoscopy could form an indispensable tool for most vitreoretinal surgeons, and improve patient outcomes, substantial unresolved usability issues have prevented greater incorporation of this highly valuable approach into routine retina surgery. These barriers can be overcome with a different approach (FIG. 5, FIG. 10, FIG. 11, FIG. 13, FIG. 14) to ophthalmic endoscopy than employed by current commercial systems (FIG. 3).

The present invention discloses a method for performing ophthalmic endoscopy that in one embodiment provides a hands-free (FIG. 10, FIG. 11), high resolution (193, 194, FIG. 7), top-down (FIG. 6), true stereo (FIG. 17), always in focus (FIG. 5), endoscopic wide field of view of the retina (FIG. 5, FIG. 17). This system incorporates a novel, direct camera sensor (41) module (43) enhancing image resolution (FIG. 7) and allowing for a low-cost (<$100) disposable (FIG. 4), as compared to expensive (>$5,000) commercial fiber optic based ophthalmic endoscope (FIG. 3). Real-time, artificial intelligence assisted perspective transformation (FIG. 6) and image enhancement (FIG. 7) provides this proposed high resolution, top-down view.

The present disclosure provides an ophthalmic endoscope for visualization of the retina with advantages over existing (FIG. 3) ophthalmic endoscopic approaches including:
1. Direct sensor (41) versus fiber-optic (17, 32) based visualization of the retina enabling higher resolution images: Fiber optic based images have limited image resolution on ophthalmic endoscopes, with as few as 6000 pixels in smaller gauge instruments, to at most 17,000 pixels in 19 gauge probes. In 2019 Omnivision, introduced a miniaturized (0.65 mm×0.65 mm×1.2 mm) camera module (FIG. 4, 41, 43), incorporating for the first time 40000 pixels in a device compatible with 19-20 gauge instrumentation.
2. Hands-free traditional viewing (FIG. 10, FIG. 11, FIG. 13, FIG. 14): Commercial ophthalmic endoscopes have to be held by the user (17), providing a view that moves with any hand motion, eliminating the possibility of typical bi-manual surgery as one hand holds the endoscope. Novel camera modules (104) can be incorporated into a hands-free, sclera mounted housing (FIG. 5). The viewing system, no longer incorporated into the surgeon light pipe (13) (as in commercial ophthalmic endoscopes (17)), better emulates the top-down (16), hands free, invariant view, provided by traditional surgical ophthalmic microscopes.
3. Variable hands-free widefield and macro views of the retina: A typical viewing system has both widefield and macro viewing options by swapping viewing lenses on the surgical operative microscope. Widefield approaches are needed for surgical repair of peripheral retinal detachments, while macro approaches are useful for macula surgery, such as removal of pre-retinal fibrotic membranes in diabetic retinopathy. A sclera mounted endoscopic module (FIG. 10, FIG. 11) allows for variable insertion with use of a stop (51) of the camera into the eye (0-10 mm) for both wide-angle and macro views.
4. Disposable, low-cost instrumentation (FIG. 4): Traditional ophthalmic endoscopes as non-disposable instrumentation (FIG. 1, FIG. 3) suffers from both high cost, $30K for the Endoptics E4 system, Beaver-Visitec, Waltham, MA and the possibility of infection due to difficulty of sterilization. The Omnivision camera (Santa Clara, CA) is designed as a low-cost, disposable module (<$65) (FIG. 4). The present invention in a preferred embodiment envisions a camera video to USB converter to allow standard web-cam visualization of the camera module (104) video feed, with the disposable endoscope module (43) plugged into this USB video encoder, such that only the endoscope module need be replaced between patients.
5. True stereo view ophthalmic endoscope: Current ophthalmic endoscopes provide only a low resolution 2D (191, 192) view which limits the ability to manipulate micron thick retinal tissues. When dissecting pathologic retinal membranes, fine 3-D is critical. Positionally synced camera modules (104) (FIG. 13, FIG. 14) enable stereo heads-up (FIG. 6, 16) viewing for the surgeon. In a preferred embodiment, each module inserts thru a canula (106) using a standard 19-20 g sclerotomy thru the pars plana (33). In a further aspect of this preferred embodiment advanced image processing detects (FIG. 6, FIG. 12) each camera position and positionally and frame rate fuses these two camera views into a synchronized stereo view of the retina (FIG. 17).
6. Top-down perspective transformation (FIG. 6): In an additional aspect of a preferred embodiment, advanced real-time video processing (FIG. 12) is able to reorient the view provided to the surgeon, perspective transforming the current side-view (18) provided by a sclera mounted (33) ophthalmic endoscope into a traditional top-down/birds-eye stereo view of said retina (FIG. 6) of said eye as if visualizing on an surgical ophthalmic microscope looking thru said cornea (12) of said eye.
7. Real-time AI resolution/detail enhancement (FIG. 7, FIG. 9): In an additional aspect of a preferred embodiment, image resolution provided by said camera module of said ophthalmic endoscope can be further processed by artificial intelligence based, retina specific, image enhancement algorithms to provide greater than 2× resolution upscaling, leading to a >80,000 pixel interpolated image, while also enhancing retinal fine detail.
8. Repositioning of endoscope direction to enable central and peripheral retinal viewing (FIG. 10, FIG. 11, FIG. 13, FIG. 14): In an additional aspect of said preferred embodiment, visualization of the entire retina from optic nerve to ora serrata by said camera module is enabled in said invention. While a 120° endoscopic field of view is expansive (FIG. 5, 57), it does not fully reach the edge of the retina (ora (173)) without camera repositioning. In one embodiment two or more camera modules can be linked to synchronously reposition each module (FIG. 13, FIG. 14), directing said camera modules towards more central or more peripheral retina, hands-free, without losing stereopsis. In an additional aspect of said embodiment, stereo misalignment is automatically software corrected and AI enhanced (FIG. 12), increasing depth perception (FIG. 17) in a heads-up display for said vitreoretinal surgeon.

A traditional endoscope meant for viewing of any bodily structure whether for ophthalmic or other organ system (15) visualization purposes traditionally has the endoscope contained within a structure such that it can be positioned easily by an operator usually using their hand to do so (17). Given the lack of availability of light to illuminate the organ system being viewed (15), said endoscopes often also carry their own means of illumination (18) to both illuminate the surface being viewed and also transmit the image of the surface being viewed under this illumination back to the operator for diagnostic, interventional or observational purposes. In providing such function, it must be of a small enough size (17) to be easily inserted within the end organ (15), often thru means of a trocar canula (106) or simply an incision (33), into a body cavity, and then be maneuverable enough once thru the trocar canula (106) or incision (33) to visualize different aspects of the organ surface (15) without damaging said organ surface in the process. As such, for traditional medical fields in which said endoscopes are frequently used, many years of training are often required to be able utilize this tool in a facile manner. Such training is made particularly difficult given that said traditional endoscopes only have a single viewing camera (32) to observe the organ system (15). The lack of a second camera (FIG. 11, 106) on said endoscopes makes 3D stereopsis (FIG. 17) impossible, in which case other depth cues (FIG. 4, 44, 45) must be used to ascertain position of said endoscope from said organ surface (15). Also, rotation of said endoscope (17) must be controlled to establish orientation of the endoscope relative to the organ system being viewed (15). Years of experience are need to gain the muscle memory to perform said visualization of said organ system with said endoscope, and such visualization approach often becomes the primary means by which said organ system visualization and surgical intervention for said organ system occurs.

The eye, more specifically the retina, represents a particularly unique visualization and intervention approach compared to most internal end organ systems. The eye is less than 8 cm³, with 25 mm axial length, which is a particularly small space to be operating within. A trocar canula (106) based approach is still often used to insert surgical instrumentation (11, 13) into the eye to be able to reach the retina (19) with these instruments. Illumination is also frequently provided by means of a handheld instrument that utilizes fiber optics to transmit light via that handheld instrument into the eye to visualize the retina (13). However, an endoscope for visualization of said retina is less frequently needed given that visualization of the retina directly thru the cornea and lens (16, 12, 14) of an eye is possible in most cases. Thus, for most vitreoretinal surgeons, endoscopy is not a frequently practiced approach for visualization of the retina (15) and operative repair of said end organ system. Thus, many years of training are not devoted by most vitreoretinal surgeons to understanding the intricacies of use of a handheld endoscope (17) with regards to positioning in the eye and ascertaining orientation of the endoscope within the eye (18). Vitreoretinal surgeons as such expect their visualization of the surgical field to not be moving and rotating as would occur with the movement of a handheld endoscope (17).

In one preferred embodiment of the present disclosed invention the endoscope camera module (104) would not be on the end of a handheld probe, but rather be mounted to the eye (104, 106) in a set position thru means of a securing mechanism (106). Given the need for the camera module (104) of said endoscope to visualize the retina, it still must be capable of being inserted thru the side wall (33) of the eye to be able to visualize the retina (15) of said eye. A cannula (106) in one embodiment can provide such a securing mechanisms to said sclera side wall (33) of said eye. Said cannula (106) would initially be inserted initially thru said pars-plana (33) of said eye so as to not damage said retina (15) or rupture said blood vessels of said eye, and would affix itself to the eye with some means of establishing rotational orientation of said canula (106) with respect to said eye. This aspect of the invention can be particularly useful in order to provide a preferred insertion orientation of said camera module (104) with respect to said eye and said retina (15) of said eye, in order to create an appropriate non-rotated image of said retina by said camera module (104) of said endoscope. In one preferred embodiment, such orientation marker on said cannula could be a said key (52). Such a key (52) would be placed in a stereotypical position oriented with respect to said eye to allow a stereotypical orientation of said camera module (104) and camera sensor (41) to said eye (107).

A preferred embodiment of said cannula (106) would be capable of the following functions: 1) providing a path thru which said camera module of said endoscope can be inserted into said eye (FIG. 13 106, 104) 2) securing said camera module of said endoscope to said sidewall (33), sclera of said eye (33), holding said camera module during typical movements of the eye as required during vitreoretinal surgery, but at the same time not preventing said camera module (104) from being removed from said cannula (106). Said camera module (104) should be able to be removed from said cannula (106) with some force, but not requiring excessive force that could damage said cannula (105), said camera module (104), or said eye (107). In a preferred embodiment, said camera module (104) would have a similar key (52) to allow stereotypical insertion orientation of said camera module (104) into said cannula (106).

In one embodiment, said camera module (104) would be prevented from being inserted too far into the eye, by placement of a stop (51) on the end of said camera module (104) that mechanically prevents it from being inserted further into said cannula (106) and thus into said eye. In an alternate embodiment, more than one stop could be present on said camera module to allow for different pre-set insertion depths in a stereotypical manner. Such insertion depths could be provided by means of ridges along the enclosure of said camera module that would provide increased friction for insertion when said ridges touch the edges of said cannula at predetermined insertion depths. Alternate embodiments would include placing markings along said camera module (104) for preset insertion depths thru said cannula (106). In a preferred embodiment, said preset insertion depths would allow for differing field of view to provide the vitreoretinal surgeon different effective magnifications of said retina and thus effectively different imaging resolutions of said retina provided by said endoscope camera module. In a preferred embodiment said preset insertion depths would provide 150 degree (221), 120 degree (57), and 60 degree fields of view, allowing for far peripheral viewing of the retina (150 degree) for surgeries such as retinal tears or retinal detachments and also macro viewing (60 degree) of the macula (58) for surgeries such as macular hole or epiretinal membrane removal.

Said canula (106) for said insertion of said endoscope and means of securing said endoscope to the sclera (33) of said eye should be easily removeable from said eye (107) when need for retinal visualization is finished and the incision thru which said cannula (106) is placed in said sidewall (33) of said eye be either self-sealing, or easily closeable thru the typical means of placing a suture thru such incision in said sidewall (33) of said eye. In one embodiment, said cannula can be placed thru said sidewall of said eye by means of a trocar insertion blade (44) that travels thru said orifice of said cannula (106). In said embodiment said trocar insertion blade (44) is inserted thru said sidewall (33) of said eye, and then said cannula (106) is moved down said trocar insertion blade to place it thru the sidewall (33) of said eye. Subsequently said trocar insertion blade (44) is removed from said orifice of said cannula (106), leaving the orifice open for insertion of said camera module (104) thru said cannula, and leaving said cannula in place, with said cannula secure in said insertion thru said sidewall of said eye, not easily removed without force applied to said cannula. In an alternate embodiment, said cannula (106) may have said additional means of securing said cannula to said sidewall (33) to prevent disinsertion from said sidewall of said eye and also prevent rotation of said cannula inside said incision of said sidewall of said eye. In said alternate embodiment, said securing means would be able to be released in some manner to allow said cannula to be disinserted from said sidewall incision of said sclera of said eye. Such a securing mechanism may take many possible embodiments including altering size of said cannula, shape of said cannula, friction of said cannula, and suction of said cannula with respect to said sidewall of said eye. In a preferred embodiment, said additional securing mechanism should be able to be activated from the edges of said cannula once first inserted to hold said canula in place and then retracted to the edges of said canula and reset to allow said cannula to transition from a state in which high securing force is enabled with said sidewall of said eye, to a state where high securing force is disabled with said sidewall of said eye to allow for easy disinsertion of said cannula from said sidewall of said eye.

Dimensionally said cannula (106) and said camera module (104) must be compatible for standard vitreoretinal surgery. As such the size of the incision required for said sidewall (33) of said eye should not in a preferred embodiment exceed 19 gauge. Further, when said camera module (104) is in said cannula (106), the combination of said canula and said camera module should prevent leakage of intraocular fluid thru said cannula or around said cannula, and out of said eye. This prevents said cannula/camera module from causing said eye to not hold adequate pressure within said eye that allows said eye to remain inflated during said endoscopic visualization or vitreoretinal surgical procedure.

Said interaction between said cannula (106) and said camera module (106) should further in a preferred embodiment not allow said camera module (104) to easily rotate within said cannula. If said camera module can easily rotate, it would be possible that said orientation of said camera module could alter with respect to said eye, that would cause perspective correction of said image to an upright orientation to be more difficult or otherwise disorient said vitreoretinal surgeon from said spatial orientation of said endoscope camera module inserted in said cannula with respect to the orientation of said retina of said eye. The aforementioned key (52) on both said cannula and said camera module can provide a means of orienting both canula and camera module to said eye, and preventing said camera module from rotating within said canula.

Said camera module (106) in a preferred embodiment would be capable of a widefield view of the retina, defined as greater than 60 degrees field of view. In a further preferred embodiment, said widefield view of said retina of said eye would be capable of up to 160 degree field of view. Such a wide field of view would allow in this preferred embodiment full visualization of the retina from ora to ora (173) without requiring movement of said camera module (104) within said eye. Further, in a preferred embodiment using dual camera modules (FIG. 10, 11 104), the ability of both camera modules (104) to visualize the entire retinal surface (15) would enable the potential for 3D stereopsis across the entirety of the retina of said eye. This is provided for because with this field of view both cameras can see the entirety of the retina, providing two views of each point in the retina (171, 172), and thus allowing for stereopsis to be generated from these two images. 3D stereopsis (FIG. 17) allows for accurate gauging of instrumentation depth and spatial orientation within said eye.

The camera module field of view (57, 171, 213) is established by the lensing system (206) of said endoscope camera module (104). Said lensing system can be designed so as to gather light from a predefined field of view and focus said light in said camera sensor (41) of said camera module (141). In a preferred embodiment said camera lensing system (206) would be designed to visualize up to 150 degree (213, 221, 206) field of view to allow for edge to edge visualization of the retina without reorienting said camera module (104) with respect to said eye to visualize more central (58) or peripheral retina (173). In an alternate embodiment, multiple camera modules (FIG. 21) having different fields of view may be used and inserted into said cannula. In this embodiment, if more peripheral retina (173) needs to be visualized then a camera module with a larger field of view (i.e. 150 degree field of view) is inserted thru said cannula (106). If more central retina (58) needs to be visualized then a camera module with a smaller field of view (i.e. 60 degree field of view) is inserted thru said cannula (106).

The lensing system (206) of said camera module should in a preferred embodiment be designed such that when coupled with fiber optic illumination (FIG. 21, FIG. 20) it prevents stray light (212, 202) from said fiber optic illumination (203) from entering said lens system (206) and transmitting said stray light (212) to said camera sensor (41) of said camera module. Various design methods for said lensing system (206) can be utilized to limit said stray light transmission (212) to said camera sensor (41) including physical baffling (FIG. 20, FIG. 18, FIG. 5) from said fiber optic illumination (54, 203), changing the acceptance angle or aperture of said lensing system (206), limiting the light scatter (212, 204) from said illumination system to limit the opportunity of said stray light (212) from entering said lensing system (206).

Said lensing system (206) should in a preferred embodiment maintain physical dimensions (43) that remain compatible with traditional vitreoretinal surgery. Such lensing system (206) should be designed to focus light onto said camera sensor (41) and in a preferred embodiment keep similar physical dimensions as said camera sensor (41) in the x and y plane. In the z plane said lensing system depth (FIG. 22) should be minimized (43) to not require significant extension into said eye thru said cannula (106) in order to prevent interference of said surgical instrumentation also inserted in said eye for said vitreoretinal procedure. In a preferred embodiment, said lensing system would maintain dimensions of 0.6 mm×0.6 mm in x and y planes, to match well the dimensions of said camera sensor (FIG. 4) used in a preferred embodiment of said camera module, and also maintain a depth of 1.25 mm in said z plane to limit interference with said other surgical instrumentation in said eye.

The endoscope camera module (104) can in a preferred embodiment provide illumination (FIG. 20, 21) to said retina of said eye to more easily allow imaging of said retina of said eye by said camera module. In an alternate embodiment, said camera module would contain no illumination and consist of said camera sensor and said lensing system (43). In such an embodiment said illumination would be provided externally by said traditional handheld light pipe (13) with said fiber optic illumination. In an alternate embodiment said illumination would be provided externally to said camera module by use of a readily available chandelier illuminator inserted in the sclera of said eye thru a trocar canula (106). Separating said illumination system (54, 203, 202) from said camera module retinal visualization system (104, 43) in said endoscope camera module may in some embodiments be preferable to allow for a smaller endoscope camera module profile to allow for smaller cannulas that may be more compatible with instrument gauge requirements of traditional vitreoretinal surgery.

In other embodiments illumination and imaging would be provided by said camera module (FIG. 20). In a traditional commercial ophthalmic endoscope this is provided by an outer core of fiber optics (54, 203) to transmit illumination to said retina of said eye, with an inner core of fiber optics to transmit visualization of said retina of said eye to a camera sensor located outside of the endoscope in an external device (191, 192). In a preferred embodiment said illumination (54, 203) would surround said lensing system (206) of said camera module (104) (FIG. 18 181, 54). In a further aspect of said preferred embodiment, said illumination would consist of fiber optics (FIG. 5 54) to surround each side of said square camera sensor (41), thus limiting the dimensions and side of said enclosing sheath (53) of said endoscope camera module to the minimum possible. In an alternate embodiment, direct LEDs could be used for illumination of said retina of said eye, eliminating the need for said fiber optics in said illumination design. Spatially use of direct LEDs may be limited by the dimensional requirements of said camera module (104) to be compatible with said vitreoretinal surgery procedures, specifically requiring less than 19 gauge incision.

In a further aspect of this preferred embodiment, illumination is provided by said fiber optic bundles (203, 54) surrounding said lensing system and camera sensor, wherein light from said fiber optic bundles is reimaged by a UFO lens system and projected from said UFO lens system (202) onto said retina of said eye over a wide field of view (204). Said fiber optic bundles (203) have an optically limited acceptance and transmission angle. Without re-imaging light from said fiber optics bundles (203), said light would not be able to evenly illuminate a 150 degree field of view (221). Said UFO lens accepts said light from said fiber optic bundles and spreads said light uniformly over said wide-field of view imaged by said camera module (104). In a further preferred embodiment of said illumination (FIG. 20, 21) of said retina provided by said camera module (104), said illumination beam pattern (221, 210) is designed such that camera 1 (FIG. 21) receives light reflected off the retina illuminated by camera 2, while camera 2 receives the light reflected off the retina illuminated by camera 1 (FIG. 21). Such a configuration can improve illumination efficiency of said illumination design (FIG. 20, FIG. 21), providing a video stream of the retina by said camera modules that has improved brightness and contrast. Said brightness and contrast of said video stream is improved by limiting stray light (212) that eminates from said illumination design. In said embodiment, position of camera 1 relative to camera 2 (FIG. 21) would be pre-specified so that said illumination design could be optimized for these specific relative camera positions.

A single camera module (104) can only provide the viewer a non-stereoscopic view of said retina of said eye due to lacking a second camera perspective of said retina. In a preferred embodiment two (dual) camera modules (104) would be placed in said eye thru said cannulas (106) (FIG. 10, FIG. 11, FIG. 13, FIG. 14). For such a configuration two video streams are provided by two camera modules. Alignment of said video streams can provide said viewer of said video streams a 3D stereoscopic view of said retina of said eye. Said 3D stereoscopic view (FIG. 17) of said retina provides depth information to said viewer to allow said viewer to determine relative depth of instrumentation (11, 13) in said eye relative to said retina.

In one embodiment said dual camera modules (104) may be placed at any position around the circumference of the pars plana (33) relative to one another. In this embodiment symmetric placement of the dual camera modules in said eye (FIG. 10, FIG. 11) is not strictly required. In this embodiment the video streams of said camera modules would be separately processed using the known geometry of the eye, as well as constrained possible positions of said dual camera modules relative to one another and relative to the retina. Common features would be identified in said video streams which may consist of one or more of blood vessels, instruments in the eye, optic nerve, choroidal landmarks, reflection patterns from said camera module illumination. Identification of common landmarks in said video streams would allow said geometric relation of said camera modules relative to one another to be calculated (FIG. 12). Such geometric relation of said camera modules can then allow re-projection of said video streams onto a common projection plane (FIG. 17) to allow alignment of said video streams with one another to provide said viewer a 3D stereoscopic view of the retina.

In a preferred embodiment for said dual camera modules, cannulas (106) for insertion of said camera modules (104) can be placed in said sclera of said eye (33) in a stereotypical manner (FIG. 13, FIG. 14) with a defined relative position of said camera modules relative to one another (within a defined tolerance range). Such placement of said dual camera modules in said stereotypical manner can further constrain estimation of geometric position for said dual camera modules relative to one another. This constrained relative positioning simplifies the required processing of said algorithm to determine relative location of said camera modules (104) from one another and from the retina of said eye.

In a further preferred embodiment said dual camera module position can be further constrained by physically linking said camera modules by means of a linker (FIG. 13, FIG. 14, 103, 110). Said linker (103, 110) apposes said camera modules to one another in a defined geometric relationship. As said camera modules (104) or said cannulas (106) may move in said eye (107) with motion of said eye relative to said camera modules, it becomes possible that said field of view of said retina of said eye may shift due to said motion of said eye relative to said camera module. As such, said camera modules (104) or said cannula (106t) may need to be repositioned within said eye in order to maintain visualization of a particular field of view (13, 57) of the retina in said eye. The linker (103, 110) allows the repositioning of said camera modules (104) within said eye to be performed in synchrony with one another. Movement of said linker translates to coordinated movement of said camera modules to reposition their field of view relative to one another synchronously. Coordinated movement of said camera modules can further constrain geometric estimate of camera module position relative to one another, further simplifying the processing required by said alignment algorithm (FIG. 12) to perform this calculation.

In a possible alternate embodiment, one of said camera module (104) may be positioned in said sclera (33) of said eye by means of said canula (106). While one camera module would not allow for true 3D stereoscopic viewing of said retina of said eye, one camera module with canula securing camera module to sclera would allow the vitreoretinal surgeon to perform bi-manual surgery, using said video stream provided by said camera module for visualization of said retina. Said camera module (104) secured in said sclera (33) would provide a fixed field of view for said vitreoretinal surgeon, in contrast to the moving and rotating field of view when said endoscopic viewing system is incorporated into said handheld probe that is intended to also function as a light pipe (13, 17, 32). While less capable than the preferred embodiment utilizing a dual camera module (FIG. 10, FIG. 11), said one camera module would provide advantages to said vitreoretinal surgeon over said commercial fiber optic handheld endoscope (17, 32, FIG. 3). Those advantages would include improved image resolution (FIG. 7, FIG. 19), improved focal range (FIG. 4), invariant field of view more similar to usual top-down visualization (16) of the retina, wide field of view due to sclera mounted position (FIG. 5), non-rotated field of view due to sclera mounted positioning of said camera module (FIG. 5), algorithm to process said video stream to provide a top down perspective to said view of said video stream.

An essential aspect of the present invention are the described algorithms to process said dual video streams to create said top-down, birds-eye, 3D stereoscopic view of said retina of said eye. In a preferred embodiment said algorithms can perform stereo image rectification on dual camera images, segmenting optic nerve and retinal vessels to identify keypoints needed for generation of 3-D stereo-pairs. Keypoints must be identified in the video streams from said camera modules to establish 3-D landmarks in the eye and subsequent construction of a fundamental matrix to enable stereo-rectification using an algorithm based on existing image software toolboxes such as OpenCV. In this possible embodiment this process can create a stereo-pair with only horizontal disparity that can be shown to the user to provide 3-D. Map key-points can be established by segmenting camera images to extract location of blood vessels and optic nerve from each camera's video. Keypoints can then be co-aligned between dual camera module video streams. In one aspect of this embodiment of said co-alignment of keypoints, a Gabor convolution kernel with a modified FLANN matcher algorithm can perform retinal image map point extraction (FIG. 8). Said algorithm in one embodiment further comprises initial camera pose estimations based on the existing geometry of the eye, assuming a fixed 17 mm separation (FIG. 11) between said camera modules (104) inserted at the pars plana (33) of said eye. In a further aspect of the embodiment of this algorithm, the algorithm would map keypoints to 3-D landmarks and cast on consecutive images from the video stream from the same camera module to identify new keypoints and re-estimate camera module poses provided by each of the two camera modules. In a further aspect of the embodiment of this algorithm, the algorithm would calculate reprojection matrices for both images from the dual camera modules video streams, rectifying these images to a common image plane, matching 3-D landmarks on the same horizontal epipolar line. In yet a further embodiment of said algorithm, a disparity map would be calculated for rectified images using a normalized cross correlation approach. Alternate embodiments of said algorithms are possible to create a 3D stereo top-down perspective of the retina from said video streams and present to said viewer of said video streams.

In one embodiment of said algorithms to process said video stream from said camera module, the image resolution can be further upscaled to enhance visualization and retinal detail of said video stream. In said embodiment said resolution can be upscales in said video stream using an AI content aware approach to improve visualization of retinal detail of said eye. Software based, real-time upscaling of image resolution with enhancement of blood vessel detail can improve apparent image resolution to the surgeon 3× (FIG. 7). A super-resolution AI algorithm can in one embodiment be designed by training said AI using a synthetic lower resolution retinal image training dataset. Such datasets can be used for training the network to generate expected ground-truth, upscaled output. The appearance of the retina is chromatically limited, but has a stereotypical appearance of choroid, blood vessels and optic nerve. Synthetic lower resolution retinal images for training can be generated from higher resolution images derived from the operative microscope or from widefield images from other widefield higher-resolution cameras. In one embodiment an algorithm can implement a super-resolution fast convolutional neural network (eg FSRCNN (FIG. 9)) algorithm with a scaling factor 3 (200×200->600×600 pixels), with a PyTorch implementation provided via GitHub, training using this synthetic data. A FSRCNN network is capable of video 30 fps rate processing, using feature extraction, shrinking, mapping, expanding, with a final deconvolution step for mapping low resolution (LR) input image space to high resolution (HR) output image space (FIG. 9). Algorithm effectiveness can then be measured for quantitative image improvement using double-stimulus approach for subjective and no reference BRISQUE methods of input and output images for objective assessment. In this embodiment a FSRCNN has demonstrated 3× resolution real-time upscaling with qualitative improvement in image quality using subjective double-stimulus grading (FIG. 7)

In a preferred embodiment, an algorithm can remap a skewed video stream from said camera module real-time to generate a top down, heads-up perspective for retinal surgeon (FIG. 6). In one aspect of this embodiment said algorithm uses retina-specific feature points, segmentation, and optical flow to determine camera pose to sub-pixel accuracy and estimate surface geometry. The camera pose in said algorithm will be constrained: its “pan” and “tilt” will be linked to its apparent x and y position movement due to coming through a non-moving incision. Camera “roll” in said algorithm will be minimal, along with apparent z movement. In a further aspect of said algorithm correlating poses between frames are constrained mostly to 2 dimensions, a space that can be brute force searched on modern GPUs if needed, such as with CUDA with a kernel that maximizes use of the texture cache. In a further aspect of said algorithm the retinal surface geometry can be constrained to a sphere or ellipsoid. In yet a further aspect of said algorithm errors in motion tracking for the camera pose calculation can be backpropagated into the surface geometry model, adjusting the radius for example. In yet a further aspect of said algorithm, said algorithm can reproject a top-down image using estimated camera pose and estimated surface geometry. Once the camera pose and retinal surface geometry are calculated, it is a trivial matter to render them in any orientation.

### BRIEF DESCRIPTION OF THE DRAWINGS

FIG. 1. illustrates traditional vitreoretinal surgery view versus that provided by a commercially available ophthalmic endoscope.

FIG. 2 illustrates ocular conditions in which a traditional top-down view thru the cornea and lens of the eye is not possible due to opacification

FIG. 3 illustrates appearance and use of a commercially available ophthalmic endoscope.

FIG. 4 illustrates possible embodiment of novel ophthalmic endoscope using a direct imaging ultra-small camera sensor in the eye.

FIG. 5 illustrates one embodiment of a hands-free ophthalmic endoscope using a direct imaging camera sensor with fiber optic illumination.

FIG. 6 illustrates computational correction of a skewed view provided by an ophthalmic endoscope into a traditional top-down view that can be presented to the vitreoretinal surgeon.

FIG. 7 illustrates upscaling of the resolution of the image provided by the camera module using an artificial intelligence algorithm to enhance detail of the retina as seen by the vitreoretinal surgeon.

FIG. 8 illustrates identification of similar retinal landmarks on two images of the retina provided from different vantage points, allowing the two images to be computationally aligned with one another to create a 3D stereoscopic view to the vitreoretinal surgeon

FIG. 9 illustrates one possible embodiment of an artificial intelligence algorithm to enhance the resolution of the images provided by the camera module.

FIG. 10 illustrates one possible embodiment to coordinate the positioning of dual camera modules for an ophthalmic endoscope, allowing synchronous linked movement of the modules to allow easier image reconstruction and avoid discontinuities in the 3D stereoscopic image presented to the vitreoretinal surgeon

FIG. 11 illustrates an alternate embodiment to coordinate the positioning of dual camera modules for an ophthalmic endoscope, allowing synchronous linked movement of the modules to allow easier image reconstruction and avoid discontinuities in the 3D stereoscopic image presented to the vitreoretinal surgeon

FIG. 12 illustrates one embodiment for combining images from two camera modules into one or more output video streams by identifying common landmarks between images and reprojecting the two images to form for example an aligned stereo image for the vitreoretinal surgeon.

FIG. 13 illustrates more detailed version of one embodiment of a linker system to coordinate movement of two camera modules synchronously.

FIG. 14 illustrates more detailed version of alternate embodiment of a linker system to coordinate movement of two camera modules synchronously.

FIG. 15 illustrates detail design of a camera module consisting of illumination design, imaging lens design, camera sensor, and outer sheath that holes these components.

FIG. 16 illustrates zoomed out view of camera module, high level construction of camera module, and its insertion thru canula into the eye.

FIG. 17 illustrates how two camera modules positioned 1 clock hour apart are capable of generating a 3D stereo view of the retina.

FIG. 18 illustrates illumination design with imaging design to enable camera module to both illuminate and image 150 degree field of view.

FIG. 19 illustrates comparison of image quality from commercial fiber optic imaging ophthalmic endoscope as compared to image quality of one embodiment of a direct imaging camera module ophthalmic endoscope.

FIG. 20 illustrates UFO illumination lens design to spread fiber optic illumination over a 150 degree field of view

FIG. 21 illustrates principles of dual camera module illumination design

FIG. 22 illustrates lensing system design for camera module in dual camera ophthalmic endoscope wherein said lens system design images a 150 degree field of view onto said camera sensor of said ophthalmic endoscope module.

### DETAILED DESCRIPTION OF THE DRAWINGS

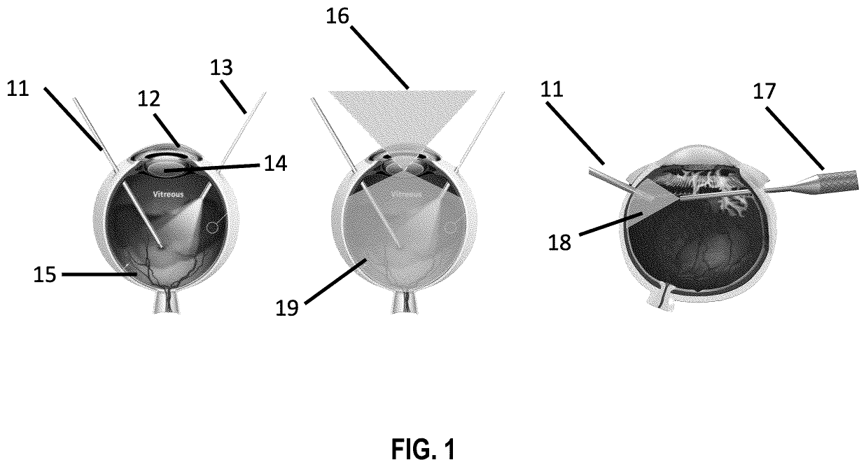

FIG. 1 Comparison of standard top-down down (16) and endoscopic narrow side view (18) for vitreoretinal surgery. Vitreoretinal surgery is traditionally performed using two bi-manual ports (11, 13), one providing illumination to the retina (15) via a fiber optic light pipe (13) and the other providing the ability to cut vitreous and fibrotic scar tissue using a guillotine vitrector (11). Visualization occurs thru the cornea (12) and lens (14) of the eye, with optics on the surgical microscope providing a top-down (16), hands-free wide field of view (19). In contrast, commercial endoscopes (17) bypass the cornea and lens allowing fiber optic based visualization of the retina using a handheld endoscopic probe (17). While useful when lens and cornea are opacified 1) bi-manual surgery is impossible due to having to hold the endoscope (17) 2) the field of view is much narrower (18) and if the probe is rotated, becomes skewed 3) stereopsis is lost

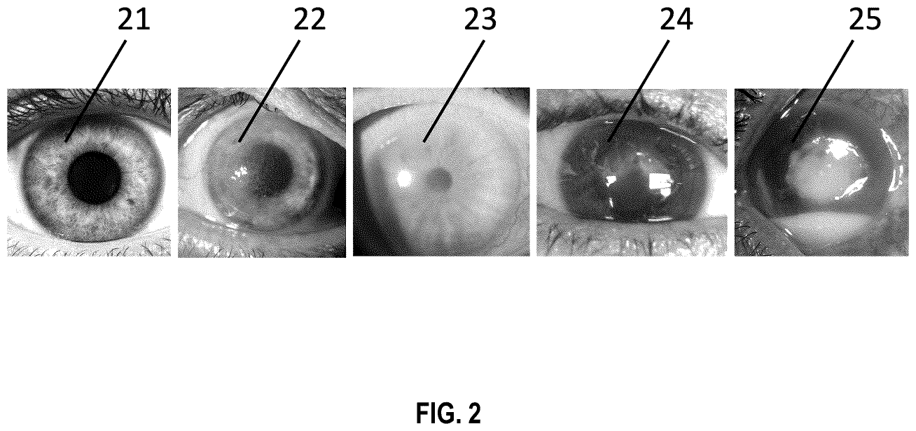

FIG. 2 Obscuration of top-down surgical view by opacification of cornea and lens. (21) A traditional vitreoretinal surgical approach requires a clear cornea and lens to observe the retina thru. (22), (23) Pre-operatively or even during surgical procedures themselves the cornea can frequently become opacified with cornea edema and Descemet's folds. (24) Trauma can cause both corneal edema and cloud the lens of the eye. (25) Endophthalmitis, a surgical emergency, can completely obscure the cornea, requiring an endoscopic approach to surgical intervention.

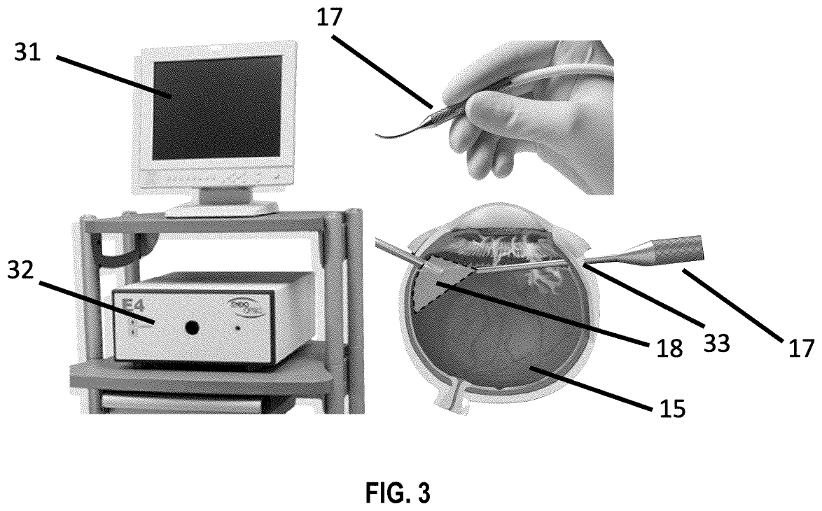

FIG. 3 Commercially available EndoOptiks E4 endoscope (17, 32). illumination and camera sensor are located in an external unit (32), with endoscope fiber optic probe (17) connected to this unit, requiring manual focusing and verification of correct up-down orientation of probe before insertion into the eye. An LCD flat screen (31) is provided for 2D viewing of the transmitted image. Both illumination to the retina (18) and image recorded by the camera module from the retina (32) are transmitted via fiber optics in a handheld straight or curved probe (17). Image resolution limited to 600-17000 pixels depending on probe gauge (17). The probe is inserted thru the sclera (33) at the pars plana of the eye and directed at the area of interest of the retina (15) and illuminated by fiber optic illumination (18) over this area (18), providing a magnified and limited side rather than standard top-down wide field of view.

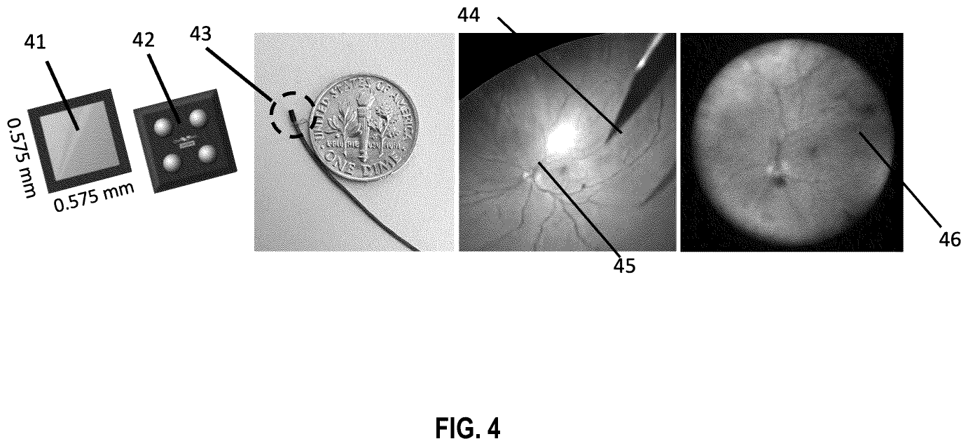

FIG. 4 Use of a ultra-small sensor (41) for direct endoscopic viewing of the retina. Commercial ophthalmic endoscopes have used external (off instrument) camera sensors (with image transmitted by fiber optics) limiting image quality and resolution to 6-17K pixels. Omnivision provides a 0.575×0.575 mm sensor (41) with 40K pixels. A 4 pin interface provides power and data transmission to the sensor (42). An initial demonstration module (42) 0.65 mm wide, with 120° FOV camera demonstrates the feasibility of direct sensor rather than fiber optic based imaging of the retina. Improved resolution of the retina on a model eye (45) compared to resolution from commercial endoscope (46) and wide focal range 3-25 mm allowing instruments (44) and retina (45) to be always in focus.

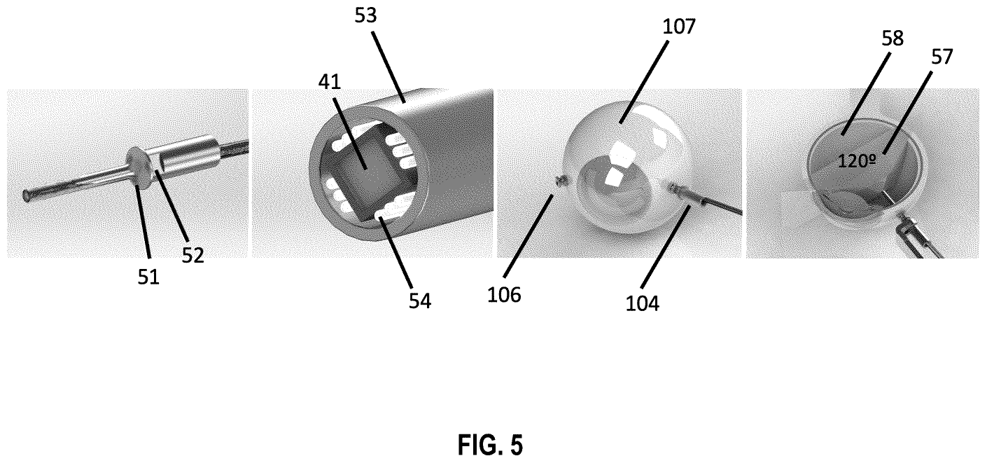

FIG. 5 Construction of a hands-free endoscopic imaging module (104) using Omnivision camera (41). The Omnivision 150° field of view (FOV) imaging module (41) is surrounded by fiber optics (54) designed to provide uniform illumination over the retinal area imaged. A metal outer sheath (53) contains module (41) and fiberoptics (54) and is keyed (52) to indicate camera orientation. The camera module (104) is inserted thru a custom form-fitted cannula (106) (also keyed), providing friction to insertion to prevent camera motion, with a stop on the camera module (51) preventing insertion more than 10 mm into the eye. Use of direct camera sensor (41) allows up to 40K pixel, 150 FOV (57) imaging (conventional endoscope is 17K pixel), reducing to 60° FOV when the camera module (104) is fully inserted 10 mm into eye up to the stop (51), providing a magnified macro view of macula (58) for fine retinal tissue manipulation near the center of the vision.

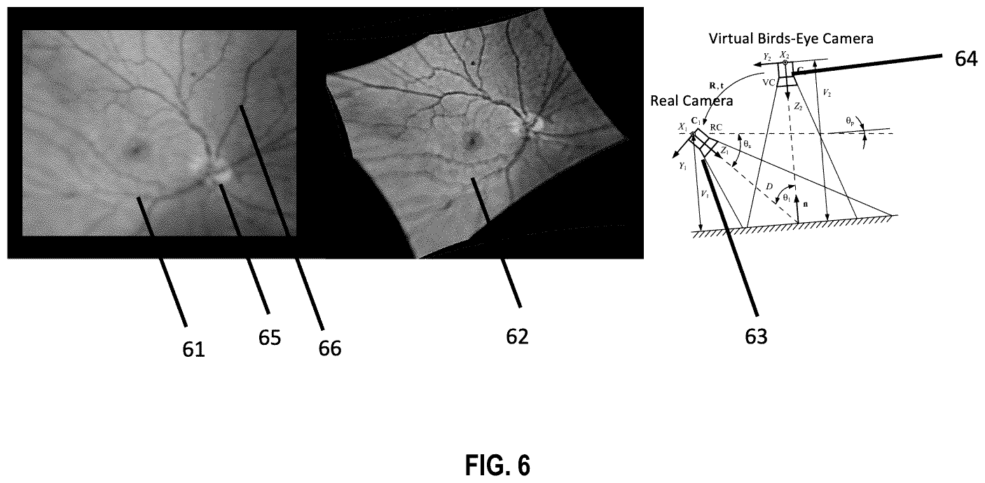

FIG. 6 Correction of endoscope skew (63) to provide a standard top-down surgical view (64). A traditional endoscope due to angled insertion with respect to the retina will provide a skewed and rotated image (61). Knowing the angle, distance, and magnification of the endoscope relative to the retina of the eye (63, 64) and identifying retinal landmarks including the optic nerve (65) and retinal blood vessels (66), allows this perspective to be topologically remapped to a more traditional top-down perspective (64) as provided by a surgical microscope viewing system. The skewed and rotated view provided by ophthalmic endoscope (61, 63) A remapping to the same view that would be provided if the surgeon was looking top-down thru cornea and lens (64). Perspective remapping automatically and real-time at 30 frames per second allows the surgeon to reconnect their hand movements with image perception.

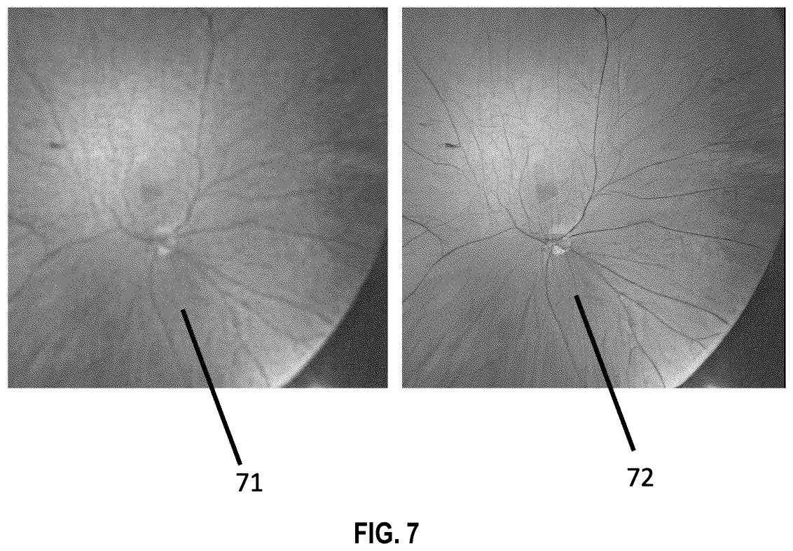

FIG. 7 Single video frame enhancement to improve endoscopic retinal visualization. Single frame (71), 40K pixel image, from 30 fps video under very low-light conditions with motion to purposefully introduce high video signal noise. 4× upscaling enhancement of image resolution (72) and clarification of retina detail for this single frame using a content-aware algorithm. Artifact removal, and GPU accelerated processing enable real-time enhanced visualization of the retina.

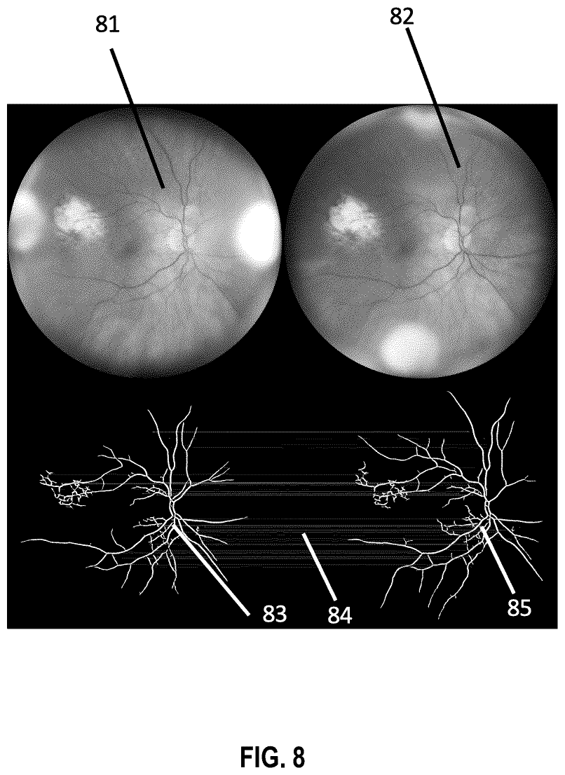

FIG. 8—Automated blood vessel detection and segmentation with detection of interframe shift on consecutive widefield images Gabor convolution kernel used to skeletonize blood vessels (83, 85) on two images (81, 82) with slightly shifted field of view and perspective and then detect matching feature points (84)

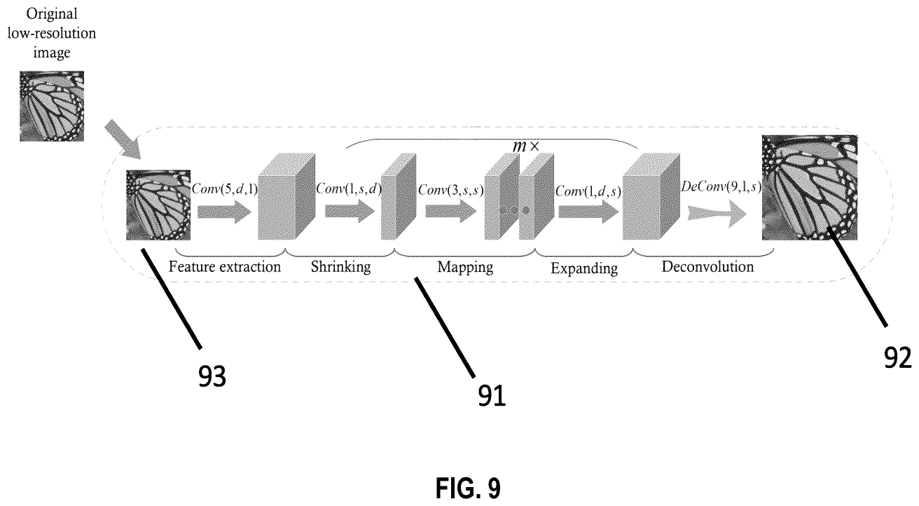

FIG. 9 FSRCNN (91) is a variant of convolutional neural network approach for image upscaling with the algorithm proven capable of real-time (<3 ms) per frame processing. A synthetic dataset is created from degraded widefield operative microscope images for network training, targeting 4× upscaling of the image resolution from an original 200×200 pixels (93) to 800×800 pixels (92). Upscaling image resolution improves blood vessel, optic network, and fibrotic tissue definition for fine surgical manipulation.

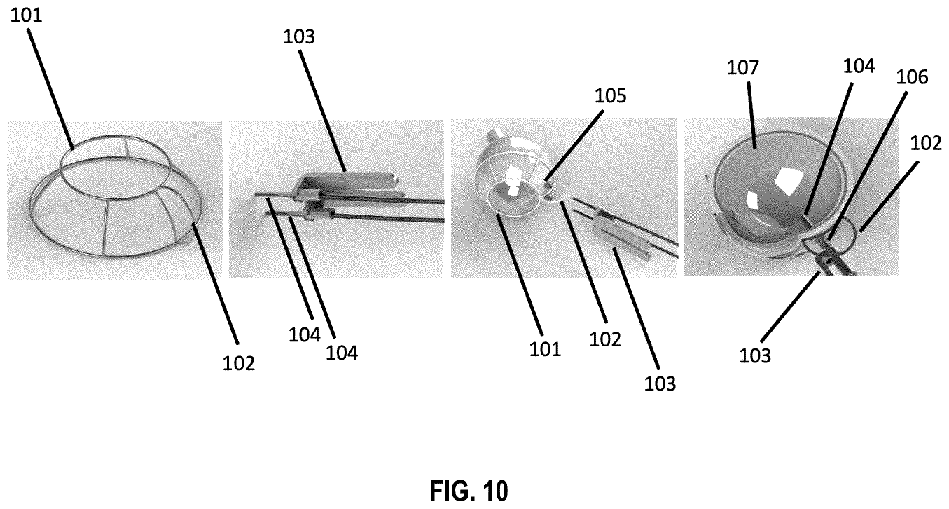

FIG. 10 Achieving stable tilt for stereo endoscopic 2-camera system to observe peripheral-most retina. Modified McNeil Goldman fixation ring (101) is placed around the eye with a single loop (102) between two camera modules (104) A camera module linker with tilt handle (103) joins two camera modules separating them by 5 mm The tilt handle (103) on the linker is slotted onto the fixation ring loop (102) between the cannulas (104). Camera module are inserted thru cannulas (106). Cutaway of loop (102) and tilt handle (103). Tilt is achieved by rotating the tilt handle (103) along the fixation ring loop (102). Friction between loop (102) and tilt handle (103) holds the tilt handle in place, maintaining set tilt. Fixation ring (101) rotates with the eye (107), so that the camera modules (104) inserted into cannulas (106) maintain set tilt and field of view, even with eye (107) rotation.

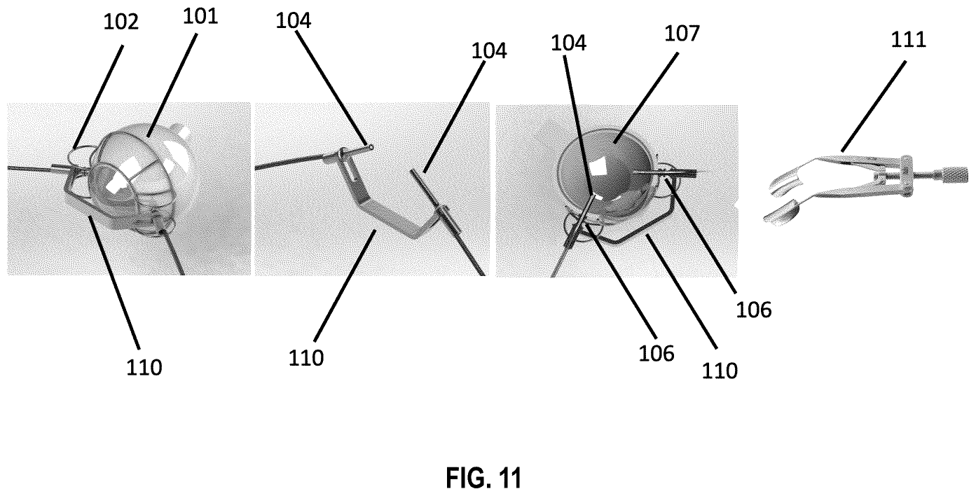

FIG. 11 Alternate embodiment for linker (110) when camera modules (104) have further separation from one another than FIG. 10. Fixation ring (101) is again placed around the eye (107) with a double rather than single loop (102) surrounding each cannula (106) Linker (110) placed after camera modules (104) are inserted in canula, synchronizes motion between the two camera modules (104) Linker cross connecting to the double loops (102) allows up/down tilt of the camera modules (104). Eye speculum (111) can be modified to attach to linker (110) to coordinate up down tilt of camera modules (104).

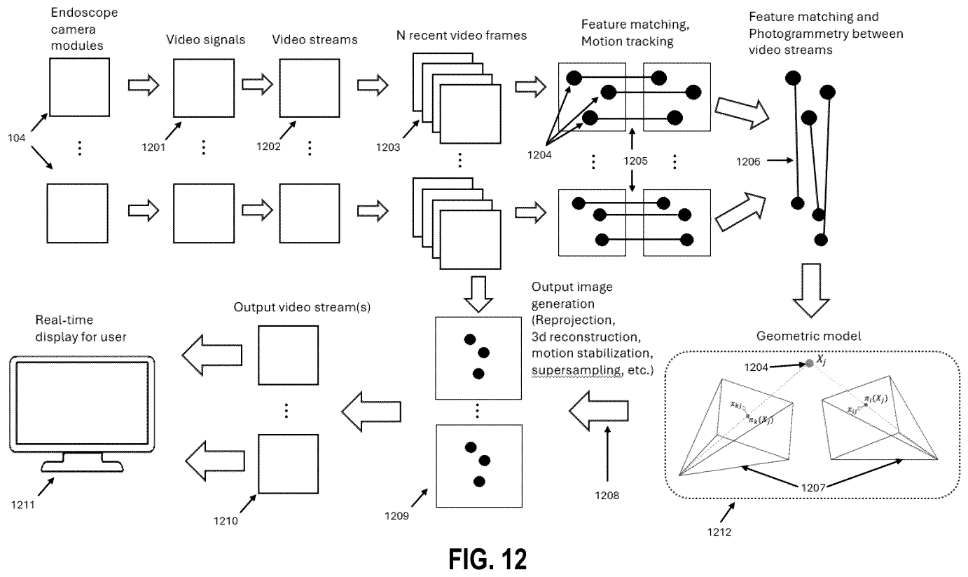

FIG. 12 Diagram of taking input video signals received from two or more Endoscope camera modules (104) to create one or more video stream sent to a display such as a monitor, lenticular display, VR headset. Input video signals (1201) are received from endoscope camera modules (104), and decoded into video streams (1202). A buffer of N recent video frames (1203) is kept in memory for each video stream. Feature points (1204) are detected using e.g. SIFT, and matched across recent video frames (1203), producing a per-frame feature matching (1205) for each video stream (1202). Features (1204) are also matched between different video streams and their respective video frames (1203), producing a feature matching (1206) which can be fed to a photogrammetric algorithm like RANSAC to produce a geometric model (1212). This geometric model (1212) could be 2d, a simple reprojection with matching of apparent 2d motion like translation, rotation and scaling, such as when the endoscope camera modules are linked together and presented to the user as a stereo pair. This geometric model (1212) could also be a 3d model of camera geometry (1207) and 3d feature points (1204), in the case of 3d model reconstruction and/or view synthesis. Output image generation (1208) then combines the geometric model (1212) and recent video frame(s) (1203) to create output image (1209) to be assembled sequentially into video stream(s) (1210) to be displayed to the user.

The process of output image generation (1208) includes at least a step of motion stabilization which could take a few forms. One form of motion stabilization is simple reprojection of input video frames (1203) which corrects for rotation of the camera. Another form which implicitly includes motion stabilization, is 3d reconstruction and view synthesis, where views are synthesized from a constant user-defined position such as top down.

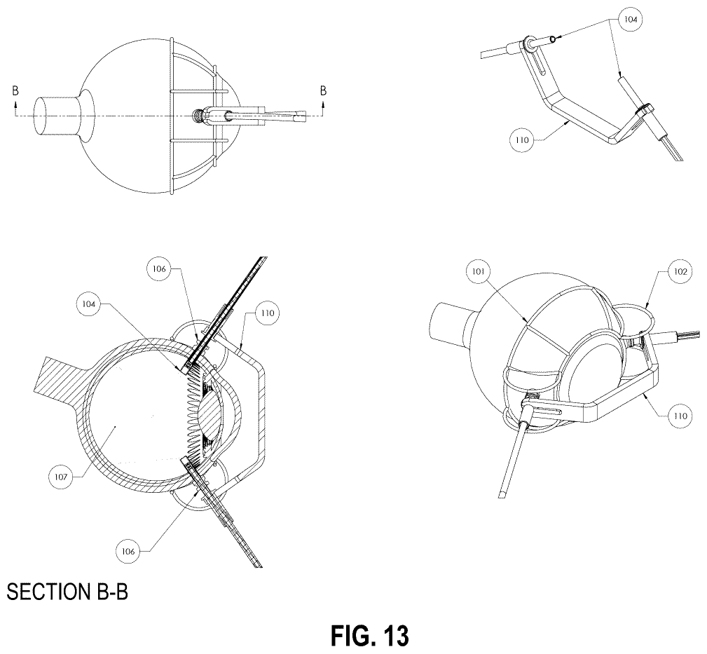

FIG. 13 Embodiment of linker design to coordinate motion of dual camera modules in ophthalmic endoscope. Linker (110) spans two camera modules (104) inserted thru cannulas (106) positioned in said model eye (107). Fixation ring (101) contains two loops on each side of the fixation ring (102), between which the camera modules (104) and cannulas (106) are situated on said eye. Cannulas are inserted at the pars plana of the eye. When linker (110) is moved along its longitudinal axis is moves the two attached camera modules (104) in synchrony with one another, maintaining their relative position to one another. The linker (110) also allows for more stereotypical placement of camera modules from one another, simplifying computational modeling of the relative view provided by the two camera modules to one another. This allows faster renders of positional transformation when constructing a 3D stereo top-down view from said images of said dual camera modules (see FIG. 12).

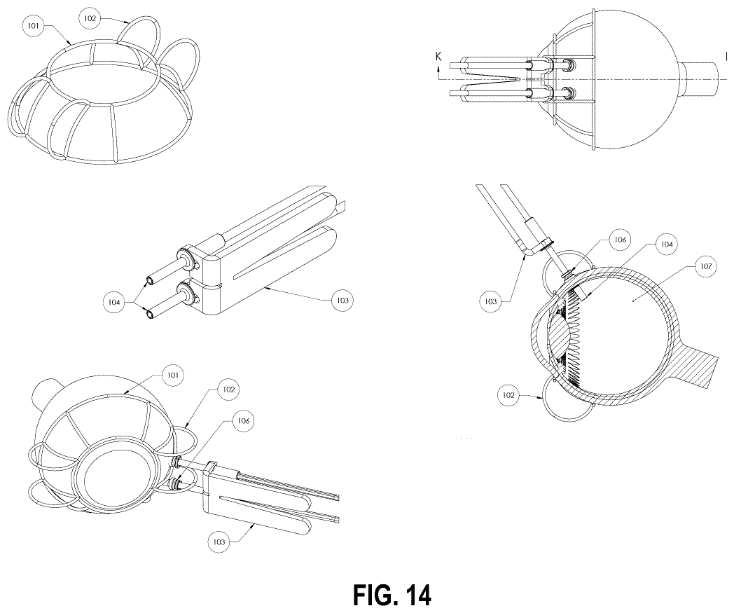

FIG. 14 Alternative embodiment to coordinate movement of endoscope dual camera modules (104) with one another. Tilt handle (103) connects two camera modules (104) together, placing them one clock hour apart on a stereotypical eye (107). Fixation ring (101) is placed on the eye, and camera modules (104) are inserted thru cannulas (106) located in the pars plana of the eye (107). Tilt handle (103) is aligned with the loop (102) on said fixation ring, such that the loop provides friction for the tilt handle and also provides a surface along which said tilt handle is able to mode in an anterior to posterior tilt motion to move said camera modules (104) in synchrony with one another. The tilt handle (103) links motion of the two camera modules (104) so that they can be moved coordinately to view more central retina or more peripheral retina as required by the vitreoretinal surgeon. The stereotypical separation can be modeled more easily to enable easier computation of the perspective transformation needed to provide the vitreoretinal surgeon a top-down 3D stereoscopic view.

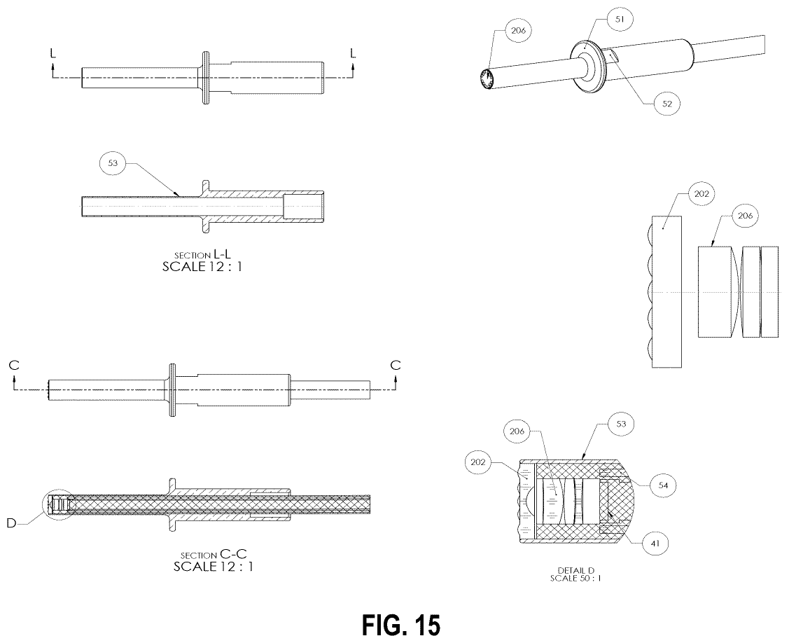

FIG. 15 One embodiment of camera module (104). Camera module (104) consists of an outer metal sheath (53) containing camera sensor (41), imaging optics (206), with imaging optics surrounded by fiber optics (54, 202). The fiber optic bundle (54), launches into a UFO lens (202) which evenly speeds the illumination over a 150 degree field of view. The imaging optics (206) are designed to image a 150 degree field of view, providing imaging capability to the ora serata of the retina. Fiber optics (54) are separated from imaging optics (206) in a manner designed to reduce stray light entry of the illumination to the camera sensor (41).

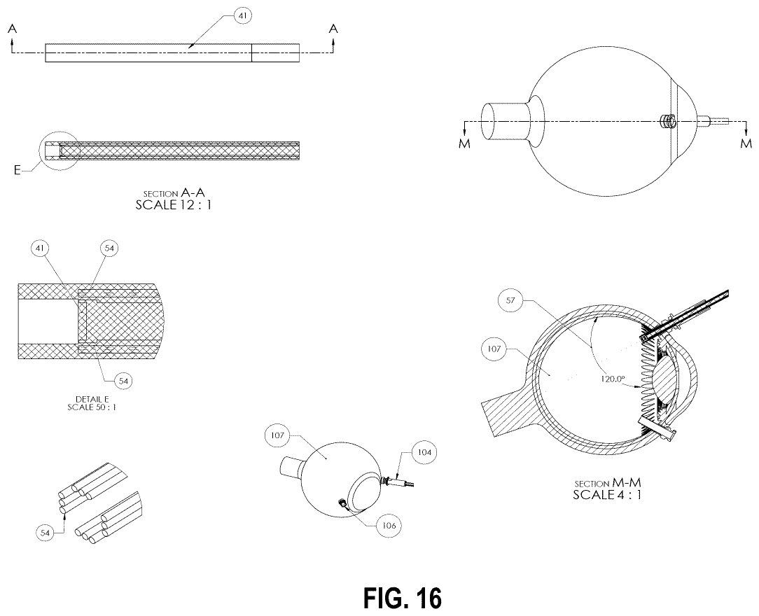

FIG. 16 Zoomed out camera module (104) assembly. Camera module (104) is inserted thru cannula (106), with cannula inserted thru the pars plana of the eye (107). Camera module (104) consists of an imaging sensor (41) surrounded by fiber optic bundles (54) that provide illumination of the retina. Imaging sensor (41) when inserted part way in the canula (2-5 mm) can image a field of view from 120 degrees to 150 degrees depending on the imaging optics (206) in front of the camera sensor (41). When the camera module (104) is inserted fully into the canula (10 mm) it can image a 60 degree field of view to provide a more magnified image of the macula for fine tissue manipulation.

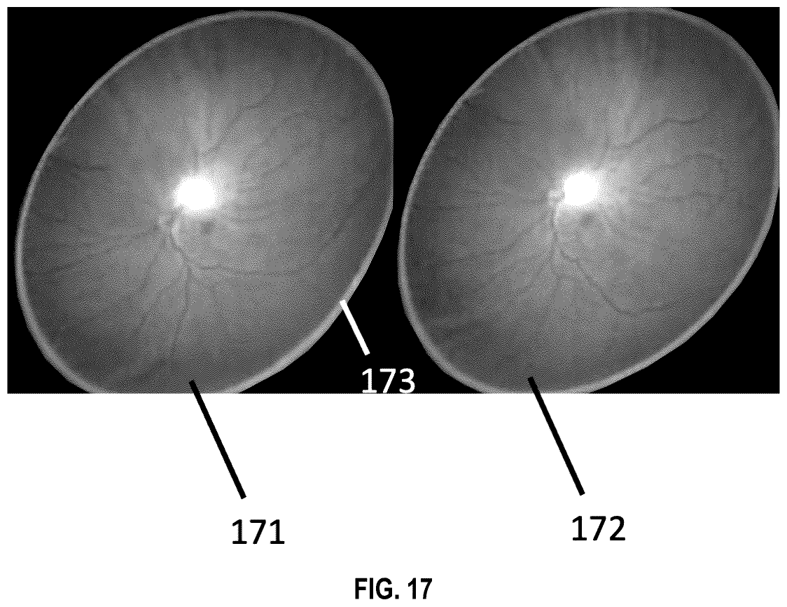

FIG. 17 Dual camera modules (104) provide separate images from each module (171, 172) with said modules positioned at said pars plana of said model eye (107), and separated by 1 clock hour. Said images (171, 172) from said camera modules provide 160° field of view able to image the entire ora serata (173—white ring) on said model eye. Thus said dual camera modules provide full stereopsis across the entire retina of said model eye without repositioning said camera modules. Stereopsis, 3D aspect provided by said dual camera modules, can be appreciated by focusing on a finger held in front of one's nose while looking at both images in the center and then removing said finger (showing a third virtual image in the middle of these two images that is in 3D stereo).

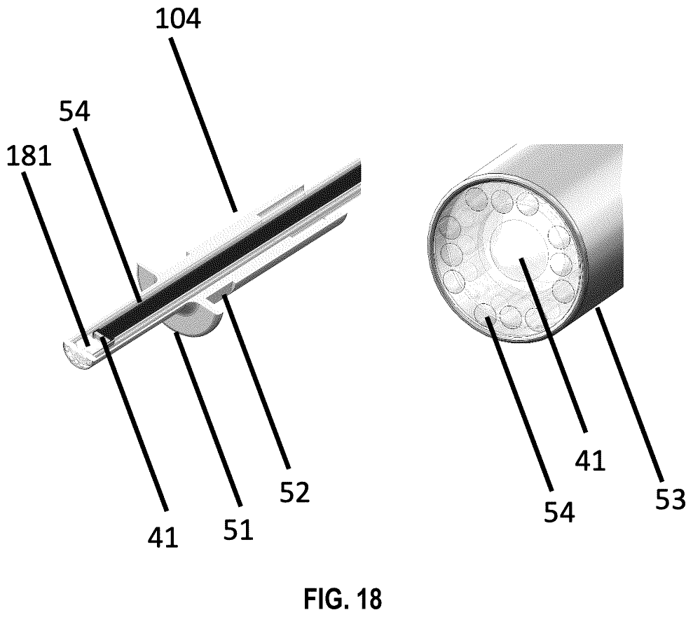

FIG. 18 A hands-free endoscopic camera module (104) using Omnivision camera sensor (41). A,B) The Omnivision camera sensor (41) is paired with 150° field of view (FOV) lens design (181) and surrounded by fiber optics that have a UFO design (54) to provide uniform illumination over the retinal area imaged by said Omnivision camera sensor (41) and said 150° field of view (FOV) lens system (181). A metal outer sheath (53) contains said camera sensor (41) and said fiberoptics (54) and is keyed to indicate camera orientation (52). Said camera module (104) has a stop (51) to prevent insertion into the eye thru said cannula (106) past 10 mm.

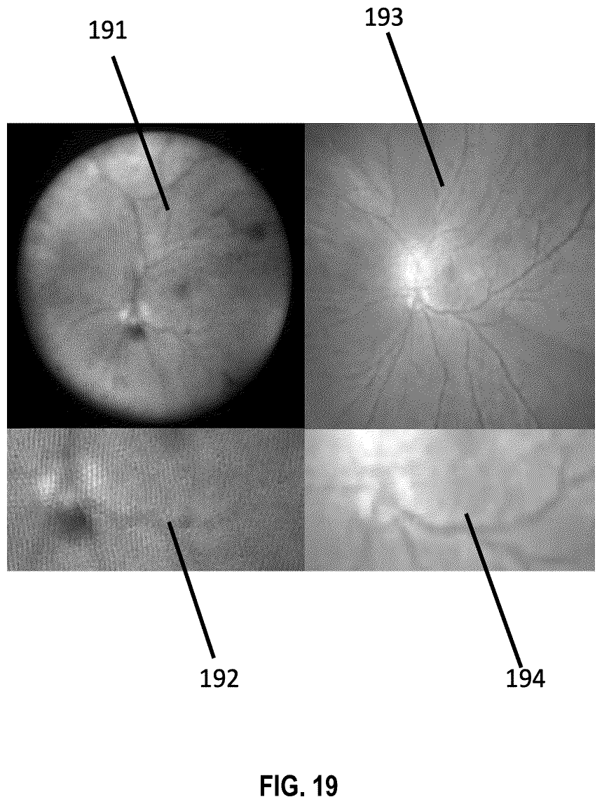

FIG. 19 Comparison of image quality from single frame full field (191) and zoomed in portion of full field (192) image of commercial EndoOptics E4 20 gauge probe (17, 32) and single frame full field (193) and zoomed in portion of full field image (194) from Omnivision sensor camera module (104), on the same model eye (107) at the same relative position from said retina of said model eye. The EndoOptics image (191, 192) has very low resolution, artifacts due to front face of fiber optics transmitting image to sensor, smaller FOV, requires 3× greater light levels to take the same exposure image as compared to the Omnivision sensor camera module (104).

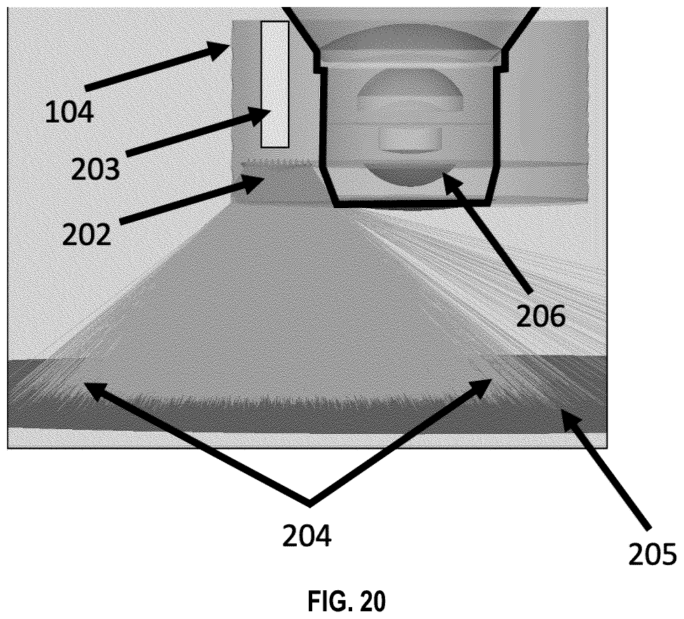

FIG. 20 UFO lens design for illumination of said retina of said eye. Front lens of the ophthalmic endoscope is fabricated in plastic, such that the edge/brim of front rim (202) images the fiber optic bundle (203) and distributes light (204) rays across the illumination surface (205). This provides a full uniform illumination of the entire field of view imaged by the camera module. Design is optimized such that image of retina on the camera sensor (41) does not also contain stray light from the illumination system (202, 203) into the lensing system (206) of the camera module (104).

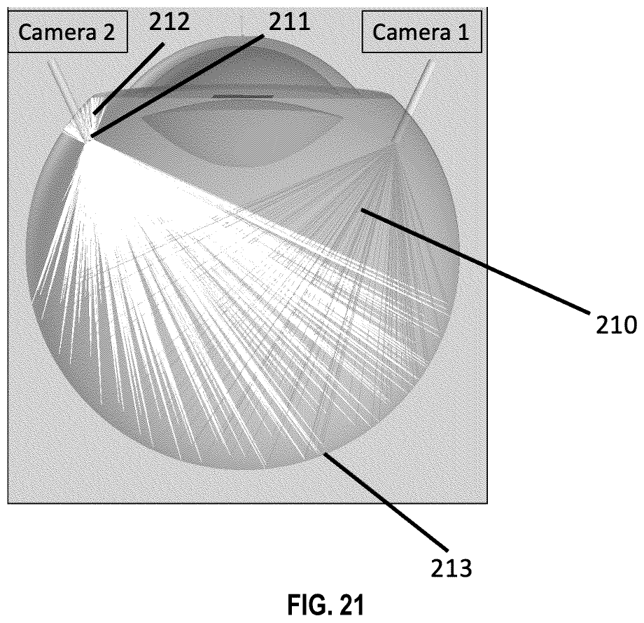

FIG. 21 Dual-camera module illumination design, with stray light analysis. Illumination (210) from camera module 1 strike the retina (213) of said eye and are reflected into camera module 2 (211), some rays do not reach the cannula and are lost as stray light (212). The illumination design of a two camera system can be optimized to improve light transmission efficiency with this approach.

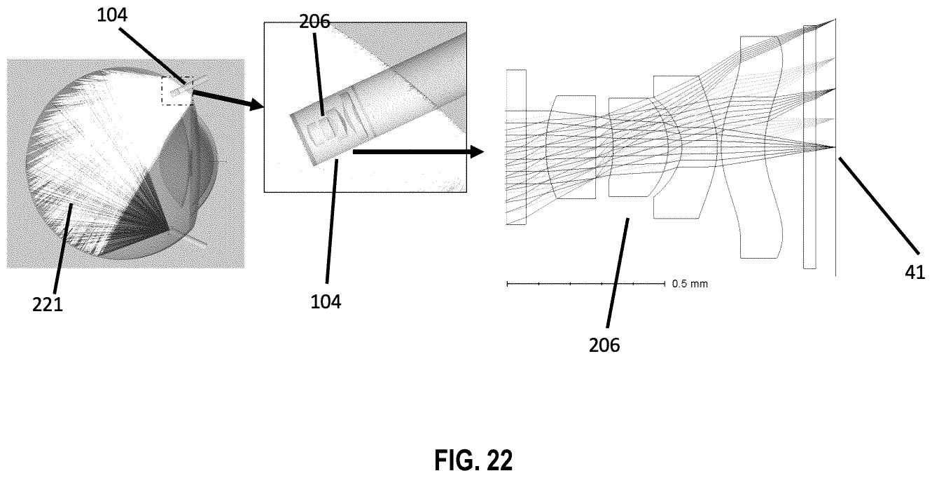

FIG. 22 Lensing system for ophthalmic endoscope camera module (206). Multi-element lensing system with axial length <1.25 mm, diameter 0.8 mm (206) provides requisite 150° field of view with required dimensions. Fiber optic illumination (54) surrounding the lensing module is coupled with a UFO lensing approach (202) to the front lens (206) to illuminate the same 150° field of view (221) that is imaged by the camera module (104, 41). Illumination is optimized to reflect rays into the sensor of the camera module (41) on the opposite side of the eye (221), while minimizing stray light into the camera sensor on the same side of the eye from whence the illumination originates.

### REFERENCES

1. Chun, D.W., M.H. Colyer, and K.J. Wroblewski, Visual and anatomic outcomes of vitrectomy with temporary keratoprosthesis or endoscopy in ocular trauma with opaque cornea. Ophthalmic Surg Lasers Imaging, 2012. 43(4): p. 302-10.
2. Martiano, D., G. L'Helgoualc'h, and B. Cochener, [Endoscopy-guided 20-G vitrectomy in severe endophthalmitis: Report of 18 cases and literature review]. J Fr Ophtalmol, 2015. 38(10): p. 941-9.
3. Ren, H., et al., [Evaluation of endoscopy assisted vitrectomy for the treatment of severe traumatic eyes with no light perception]. Zhonghua Yan Ke Za Zhi, 2014. 50(3): p. 194-6.
4. Ciardella, A.P., et al., Endoscopic vitreoretinal surgery for complicated proliferative diabetic retinopathy. Retina, 2001. 21(1): p. 20-7.
5. Ajlan, R.S., A.A. Desai, and M.A. Mainster, Endoscopic vitreoretinal surgery: principles, applications and new directions. Int J Retina Vitreous, 2019. 5: p. 15.
6. Norris, J.L. and G.W. Cleasby, An endoscope for ophthalmology. Am J Ophthalmol, 1978. 85(3): p. 420-2.
7. Wong, S.C., T.C. Lee, and J.S. Heier, 23-Gauge endoscopic vitrectomy. Dev Ophthalmol, 2014. 54: p. 108-19.
8. Marra, K.V., et al., Indications and techniques of endoscope assisted vitrectomy. J Ophthalmic Vis Res, 2013. 8(3): p. 282-90.
9. Yeo, D.C.M., et al., Endoscopy for Pediatric Retinal Disease. Asia Pac J Ophthalmol (Phila), 2018. 7(3): p. 200-207.
10. Kawashima, S., M. Kawashima, and K. Tsubota, Endoscopy-guided vitreoretinal surgery. Expert Rev Med Devices, 2014. 11(2): p. 163-8.
11. Eckardt, C. and E.B. Paulo, HEADS-UP SURGERY FOR VITREORETINAL PROCEDURES: An Experimental and Clinical Study. Retina, 2016. 36(1): p. 137-47.
12. Charles, S., The Future of Surgical Retina in the Era of Medical Retina. Retinal Physician, 2017. 14: p. 23-25.
13. Venincasa, M.J., et al., Perceptions of Vitreoretinal Surgical Fellowship Training in the United States. Ophthalmol Retina, 2019. 3(9): p. 802-804.
14. Uram, M., Ophthalmic laser microendoscope endophotocoagulation. Ophthalmology, 1992. 99(12): p. 1829-32.
15. Ghosh, P., et al., On Localizing a Camera from a Single Image. arXiv, 2020. 2003.10664v1.
16. Yavuz, Z. and C. Kose, Blood Vessel Extraction in Color Retinal Fundus Images with Enhancement Filtering and Unsupervised Classification. J Healthc Eng, 2017. 2017: p. 4897258.
17. Samawi, H., A. Al-Sultan, and E. Al-Saadi, Optic Disc Segmentation in Retinal Fundus Images Using Morphological Techniques and Intensity Thresholding. 2020 International Conference on Computer Science and Software Engineering (CSASE), 2020.
18. Taryudi and M. Wang, 3D object pose estimation using stereo vision for object manipulation system. 2017 International Conference on Applied System Innovation (ICASI), 2017.
19. Bleser, G., H. Wuest, and D. Stricker, Online camera pose estimation in partially known and dynamic scenes. Proc. of IEEE/ACM International Symposium on Mixed and Augmented Reality, 2006: p. 56-65.
20. Dong, C., C. Loy, and X. Tang, Accelerating the Super-Resolution Convolutional Neural Network. arXiv, 2016. 1608.00367v1.
21. Wang, X., et al., Real-ESRGAN: Training Real-World Blind Super-Resolution with Pure Synthetic Data. arXiv, 2021. 2107.10833v2.
22. D. Zhou, X.S., and W. Dong, Image Zooming Using Directional Cubic Convolution Interpolation. IET Image Processing, 2012. 6(6): p. 627-634.
23. Venkatesh, M. and P. Vijayakumar, Transformation Technique. International Journal of Scientific & Engineering Research, 2012. 3(5).
24. Li, H., et al., Delving into the Devils of Bird's-eye-view Perception: A Review, Evaluation and Recipe. arXiv, 2022. 2209.05324v2.
25. Kholopov, I., Bird's Eye View Transformation Technique in Photogrammetric Problem of Object Size Measuring at Low-altitude Photography Advances in Engineering Research, 2017. 133.
26. Napieralla, J. and V. Sundstedt, Ultrawide Field of View by Curvilinear Projection Methods. Journal of WSCG, 2020. 28.
27. Du, S., S. Hu, and R. Martin, Changing Perspective in Stereoscopic Images. Technical Committee on Visualization and Graphics (IEEE), 2013.
28. Zhang, T., et al., Disparity-constrained stereo endoscopic image super-resolution. Int J Comput Assist Radiol Surg, 2022. 17(5): p. 867-875.

1. A multi-camera ophthalmic endoscope system comprising:
a. at least two endoscope camera modules, each camera module configured to produce a wide field of view of a retina of an eye and provide a video stream of the retina;
b. a canula for securing each of the endoscope camera modules to a sclera of the eye to allow viewing of the retina;
c. a computer configured to receive the video stream from the camera modules and execute instructions to process the video stream and transmit an output of the processing to a viewer, the instructions comprising
i. a first instruction set to process the video stream to identify orientation and position of the camera modules relative to one another and relative to the retina;
ii. a second instruction set to align the video stream to provide a 3D stereo view of the retina to the viewer;
iii. a third instruction set to transform said aligned 3D stereo view video stream to create a top-down perspective for said viewer such that the 3D stereo stream appears to the viewer as if the viewer is looking through a cornea of the eye at the retina.

2. The multi-camera ophthalmic endoscope system of claim 1 wherein the camera modules are configured such that the wide field of view is greater than 100 degrees.

3. The multi-camera ophthalmic endoscope system of claim 1 wherein the camera modules of said endoscope system comprises an illumination system configured to provide illumination of the retina.

4. The multi-camera ophthalmic endoscope system of claim 3 wherein the illumination system comprises a fiber optic illumination system configured to illuminate the wide field of view.

5. The multi-camera ophthalmic endoscope system of claim 4 wherein said illumination system comprises a UFO lens to illuminate the wide field of view.

6. The multi-camera ophthalmic endoscope system of claim 1 wherein the instructions are adapted to align the video streams using a Gabor convolution kernel.

7. The multi-camera ophthalmic endoscope system of claim 1 wherein the instructions are adapted to increase image resolution of said video stream.

8. The multi-camera ophthalmic endoscope system of claim 7 wherein the instructions are adapted to increase image resolution of said video stream using an FSRCNN algorithm.

9. The multi-camera ophthalmic endoscope system of claim 1 wherein the at least two endoscope camera modules each include a lensing system to achieve a 150 degree field of view.

10. The multi-camera ophthalmic endoscope system of claim 9 wherein the lensing systems each have an axial length of less than 1.25 mm and a diameter of less than 0.8 mm.

11. The multi-camera ophthalmic endoscope system of claim 10 wherein each camera module comprises a camera sensor and for each camera module, the corresponding lensing system and camera sensor being configured and arranged to provide an in-focus image on the camera sensor for any object located from 3 to 25 mm from the camera module.

12. The multi-camera ophthalmic endoscope system of claim 1 further comprising a linker to synchronously move the cameral modules.

13. (canceled)

14. A method for real-time processing of at least two input video signals each obtained by a corresponding endoscope camera module inserted into an eye and producing one or more output video streams to be displayed to a user, the method comprising:
a. decoding each of the at least two input video signals to form a corresponding input video stream, each input video signal comprising a sequence of input video frames stored in a memory;
b. for each input video streams, identifying and tracking feature points included in N of the sequence of input video frames;
c. matching common ones of the feature points in the input video streams;
d. using the common feature points and photogrammetric techniques to produce a geometric model in real-time of the endoscope camera modules, the geometric model comprising (i.) relative positions and orientations of the endoscope camera modules, and (ii.) estimations of positions of said common feature points; and
e. producing, from the geometric model and the input video frames received subsequent to the N input video frames, one or more motion-stabilized output video streams.

15. The method of claim 14 wherein the one or more output video streams comprises two or more output video streams, the method further comprising:
reprojecting two of the at least two input video streams each reprojected into a respective one of the two or more output video streams;
performing rotation, zoom, and motion stabilization on the two or more output video streams; and
displaying the two or more output video streams on a stereoscopic display.

16. The method of claim 14 further comprising a step of distance-based dehazing.

17. The method of claim 14 wherein the geometric model is three-dimensional (3d), and the method further comprises partially 3d reconstructing and reprojecting the at least two input video streams from one or more user-selected perspectives into one or more of the output video streams.

18. The method of claim 17, wherein said geometric model is produced using bundle adjustment.

19. The method of claim 14 further comprising a step of temporal super-sampling.

20. The method of claim 14 wherein the geometric model is constrained based on mechanical linkages of the endoscope camera modules with each other and with the eye.
